(19) United States
(12) Patent Application Publication
(10) Pub. No.: US 2007/0010568 A1
(43) Pub. Date: Jan. 11, 2007

(54) Substituted azabicyclo hexane derivatives as muscarinic receptor antagonists

(76) Inventors: Anita Mehta, Plainfield, IL (US); Bruhaspathy Miriyala, University, MS (US); Naresh Kumar, Haryana (IN); Jang Bahadur Gupta, Kobe (JP)

Correspondence Address: RANBAXY INC., 600 COLLEGE ROAD EAST, SUITE 2100, PRINCETON, NJ, 08540, US

(21) Appl. No.: 10/544,520

(22) PCT Filed: Feb. 7, 2003
(86) PCT No.: PCT/IB03/00416

§ 371 Date: Sep. 4, 2006

(51) Int. Cl. A61K 31/403; C07D 209/02

(52) U.S. Cl. 514/412; 548/452

(57) ABSTRACT

This invention relates to derivatives of substituted azabicyclo hexanes. The compound of this invention can function as muscarinic receptor antagonists, and can be used for the treatment of various diseases of the respiratory, urinary and gastrointestinal systems mediated through muscarinic receptors. The invention also relates to a process for the preparation of compounds of the present invention, pharmaceutical compositions containing the compounds of the present invention and the methods of treating the diseases mediated through muscarinic receptors.

### FIELD OF THE INVENTION

This invention relates to derivatives of substituted azabicyclo hexanes.

The compound of this invention can function as muscarinic receptor antagonists, and can be used for the treatment of various diseases of the respiratory, urinary and gastrointestinal systems mediated through muscarinic receptors.

The invention also relates to a process for the preparation of compounds of the present invention, pharmaceutical compositions containing the compounds of the present invention and the methods of treating the diseases mediated through muscarinic receptors.

### BACKGROUND OF THE INVENTION

Muscarinic receptors as members of the G Protein Coupled Receptors (GPCRs) are composed of a family of 5 receptor sub-types (M₁, M₂, M₃, M₄ and M₅) and are activated by the neurotransmitter acetylcholine. These receptors are widely distributed on multiple organs and tissues and are critical to the maintenance of central and peripheral cholinergic neurotransmission. The regional distribution of these receptor sub-types in the brain and other organs has been documented. For example, the M₁ subtype is located primarily in neuronal tissues such as cereberal cortex and autonomic ganglia, the M₂ subtype is present mainly in the heart where it mediates cholinergically induced bradycardia, and the M₃ subtype is located predominantly on smooth muscle and salivary glands (Nature, 1986; 323: 411; Science, 1987; 237: 527). A review in Current Opinions in Chemical Biology, 1999; 3: 426, as well as in Trends in Pharmacological Sciences, 2001; 22: 409 by Eglen et. al., describe the biological potentials of modulating muscarinic receptor subtypes by ligands in different disease conditions like Alzheimer's disease, pain, urinary disease condition, chronic obstructive pulmonary disease etc.

A review in J. Med. Chem., 2000; 43: 4333 by Christian C. Felder et. al. describes therapeutic opportunities for muscarinic receptors in the central nervous system and elaborates on muscarinic receptor structure and function, pharmacology and their therapeutic uses.

The pharmacological and medical aspects of the muscarinic class of acetylcholine agonists and antagonists are presented in a review in Molecules, 2001, 6: 142.

N. J. M. Birdsall et al. in Trends in Pharmacological Sciences, 2001; 22: 215 have also summarized the recent developments on the role of different muscarinic receptor subtypes using different muscaranic receptors of knock out mice.

Muscarinic agonists such as muscarine and pilocarpine and antagonists such as atropine have been known for over a century, but little progress has been made in the discovery of receptor subtype-selective compounds making it difficult to assign specific functions to the individual receptors. Although classical muscarinic antagonists such as atropine are potent bronchodilators, their clinical utility is limited due to high incidence of both peripheral and central adverse effects such as tachycardia, blurred vision, dryness of mouth, constipation, dementia, etc. Subsequent development of the quarterly derivatives of atropine such as ipratropium bromide are better tolerated than parenterally administered options but most of them are not ideal anti-cholinergic bronchodilators due to lack of selectivity for muscarinic receptor sub-types. The existing compounds offer limited therapeutic benefit due to their lack of selectivity resulting in dose limiting side-effects such as thirst, nausea, mydriasis and those associated with the heart such as tachycardia mediated by the M₂ receptor.

Annual review of Pharmacological Toxicol., 2001; 41: 691, describes the pharmacology of the lower urinary tract infections. Although anti muscarinic agents such as oxybutynin and tolterodine that act non-selectively on muscarinic receptors have been used for many years to treat bladder hyperactivity, the clinical effectiveness of these agents has been limited due to the side effects such as dry mouth, blurred vision and constipation. Tolterodine is considered to be generally better tolerated than oxybutynin. (W. D. Steers, et. al. in Curr. Opin. Invest. Drugs, 2: 268, C. R. Chapple et al. in Urology, 55: 33), Steers W D, Barrot D M, Wein A J, 1996, Voiding dysfunction: diagnosis classification and management. In “Adult and Pediatric Urology,” ed. J Y Gillenwatter, J T Grayhack, S S Howards, J W Duckett, pp 1220-1325, St. Louis, Mo.; Mosby. 3^{rd} edition).

Despite these advances, there remains a need for development of new highly selective muscarinic antagonists which can interact with distinct subtypes, thus avoiding the occurrence of adverse effects.

Compounds having antagonistic activity against muscarinic receptors have been described in Japanese patent application Laid Open Number 92921/1994 and 135958/1994; WO 93/16048; U.S. Pat. No. 3,176,019; GB 940,540; EP 0325 571; WO 98/29402; EP 0801067; EP 0388054; WO 9109013; U.S. Pat. No. 5,281,601. U.S. Pat. Nos. 6,174,900, 6,130,232 and 5,948,792; WO 97/45414 are related to 1,4-disubstituted piperidine derivatives; WO 98/05641 describes fluorinated, 1,4-disubstitued piperidine derivatives; WO 93/16018 and W096/33973 are other close art references.

A report in J. Med. Chem., 2002; 44:984, describes cyclohexylmethyl piperidinyl triphenylpropioamide derivatives as selective M₃ antagonist discriminating against the other receptor subtypes.

### SUMMARY OF THE INVENTION

The present invention provides substituted azabicyclo hexanes as muscarinic receptor antagonists and are useful as safe and effective therapeutic or prophylactic agents for the treatment of various diseases of the respiratory, urinary and gastrointestinal systems, and methods for the syntheses of the compounds. The present invention includes 3,6-disubstituted azabicyclo[3.1.0], [3.1.1] and [3.1.2]hexanes.

The present invention also provides pharmaceutical compositions containing the compounds, and which may also contain acceptable carriers, excipients or diluents which are useful for the treatment of various diseases of the respiratory, urinary and gastrointestinal systems.

The present invention also includes within its scope prodrugs of the compounds. In general, such prodrugs are fictionalized derivatives of these compounds which readily get converted in vivo into the defined compounds. Conventional procedures for the selection and preparation of suitable prodrugs are known to the artisan of ordinary skill in the art.

The invention also includes the enantiomers, diastereomers, N-oxides, polymorphs, pharmaceutically acceptable salts and pharmaceutically acceptable solvates, esters, N-oxides and metabolites of these compounds having the same type of activity.

The invention further includes pharmaceutical compositions comprising the compounds of the present invention, their metabolites, esters, enantiomers, diastereomers, N-oxides, polymorphs, pharmaceutically acceptable salts or pharmaceutically acceptable solvates, in combination with a pharmaceutically acceptable carrier and optionally included excipients.

Other advantages of the invention will be set forth in the description which follows, and in part will be apparent form the description or may be learnt by the practice of the invention.

In accordance with one aspect of the present invention, there are provided compounds having the structure of Formula I: 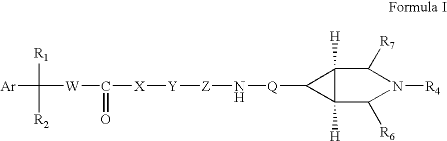
and their pharmaceutically acceptable salts, pharmaceutically acceptable solvates, esters, enantiomers, diastereomers, N-oxides, polymorphs, prodrugs, or metabolites, wherein
Ar represents an aryl or a heteroaryl ring having 1-2 hetero atoms selected from the group consisting of oxygen, sulphur and nitrogen atoms, the aryl or heteroaryl rings may be unsubstituted or substituted by one to three substituents independently selected from lower alkyl (C₁-C₄), lower perhaloalkyl (C₁-C₄), cyano, hydroxy, nitro, halogen (e.g. F, Cl, Br, I), lower alkoxy (C₁-C₄), lower perhaloalkoxy (C₁-C₄), unsubstituted amino, N-lower alkyl (C₁-C₄) amino or lower alkyl (C₁-C₄) amino carbonyl;
R₁ represents hydrogen, hydroxy, hydroxymethyl, aryl, alkylaryl amino, alkoxy, carbamoyl or halogen (e.g. fluorine, chlorine, bromine and iodine);
R₂ represents alkyl, C₃-C₇ cycloalkyl ring, C₃-C₇ cycloalkenyl ring, an aryl or a heteroaryl ring having 1-2 hetero atoms selected from the group consisting of oxygen, sulphur and nitrogen atoms; the aryl or heteroaryl rings may be unsubstituted or substituted by one to three substituents independently selected from lower alkyl (C₁-C₄), lower perhaloalkyl (C₁-C₄), cyano, hydroxy, nitro, lower alkoxycarbonyl, halogen, lower alkoxy (C₁-C₄), lower perhaloalkoxy (C₁-C₄), unsubstituted amino, N-lower alkylamino, N-lower alkylamino carbonyl (C₁-C₄);
W represents (CH₂)p, where p represents 0 to 1;
X represents an oxygen, sulphur, NR or no atom, wherein R represents H, alkyl;
Y represents no atom or CHR₅CO, methyl or (CH₂)q; wherein R₅ represents hydrogen, and q represents 0 to 4;
Z represents no atom or NHR₈CO, wherein R₈ represents (CH₂)ᵣ, wherein r represents 0 to 4;
Q represents (CH₂)ₙ wherein n represents 0 to 1;
R₆ and R₇ are independently selected from H, CH₃, COOH, CONH₂, NH₂, CH₂NH₂; and
R₄ represents hydrogen, C₁-C₁₅ saturated or unsaturated aliphatic hydrocarbon (straight chain or branched) groups in which any 1 to 6 hydrogen atoms may be substituted with the group independently selected from halogen, carbonyl, arylalkyl, arylalkenyl, heteroarylalkyl or heteroarylalkenyl having 1 to 2 hetero atoms selected from the group consisting of nitrogen, oxygen and sulphur atoms with an option that any 1 to 3 hydrogen atoms on an aryl or heteroaryl ring in said arylalkyl, arylalkenyl, heteroarylalkyl, heteroarylalkenyl rings may be substituted with lower alkyl (C₁-C₄), lower perhaloalkyl (C₁-C₄), cyano, hydroxy, nitro, lower alkoxy carbonyl, halogen, lower alkoxy (C₁-C₄), lower perhalo alkoxy (C₁-C₄), unsubstituted amino, N-lower alkylamino (C₁-C₄), N-lower alkylamino carbonyl (C₁-C₄).

In accordance with a second aspect of the present invention, there is provided a method for treatment or prophylaxis of an animal or a human suffering from a disease or disorder of the respiratory, urinary and gastrointestinal systems wherein the disease or disorder is associated with muscarinic receptors, comprising administering to a patient in need thereof, an effective amount of compounds as described above.

In accordance with a third aspect of the present invention, there is provided a method for treatment or prophylaxis of an animal or a human suffering from a disease or disorder associated with muscarinic receptors, comprising administering to a patient in need thereof, an effective amount of compounds as described above.

In accordance with a fourth aspect of the present invention, there is provided a method for treatment or prophylaxis of an animal or human suffering from a disease or disorder of the urinary system which induce urinary disorders such as urinary incontinence, lower urinary tract symptoms (LUTS), etc.; respiratory system such as bronchial asthma, chronic obstructive pulmonary disorders (COPD), pulmonary fibrosis, etc.; and gastrointestinal system such as irritable bowel syndrome, obesity, diabetes and gastrointestinal hyperkinesis with compounds as described above, wherein the disease or disorder is associated with muscarinic receptors, comprising administering to a patient in need thereof, an effective amount of compounds as described above.

In accordance with a fifth aspect of the present invention, there are provided processes for preparing the compounds as described above.

The compounds of the present invention exhibit significant potency in terms of their activity, which was determined by in vitro receptor binding and functional assays. Some of the compounds of the present invention were found to be potent muscarinic receptor antagonists with high affinity towards M₃ receptors. Therefore, the present invention provides pharmaceutical compositions for treatment of diseases or disorders associated with muscarinic receptors. Compounds and compositions described herein can be administered orally or parenterally.

### DETAILED DESCRIPTION OF THE INVENTION

The compounds described herein may be prepared by techniques well known in the art and familiar to the average synthetic organic chemist. In addition, the compounds described herein may be prepared by the following reaction sequence as shown in Scheme I.

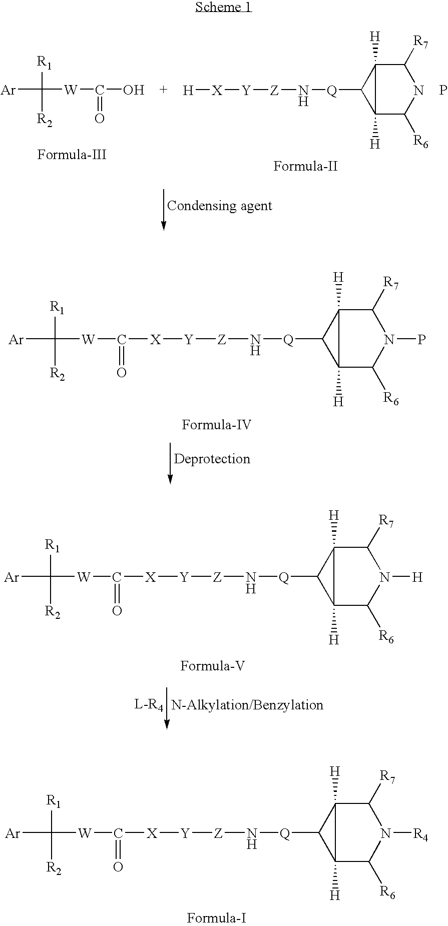

The preparation comprises condensing a compound of Formula m with the compound of Formula II wherein
Ar represents an aryl or a heteroaryl ring having 1-2 hetero atoms selected from the group consisting of oxygen, sulphur and nitrogen atoms, the aryl or heteroaryl rings may be unsubstituted or substituted by one to three substituents independently selected from lower alkyl (C₁-C₄), lower perhaloalkyl (C₁-C₄), cyano, hydroxy, nitro, halogen (e.g. F, Cl, Br, I), lower alkoxy (C₁-C₄), lower perhaloalkoxy (C₁-C₄), unsubstituted amino, N-lower alkyl (C₁-C₄) amino or lower alkyl (C₁-C₄) amino carbonyl;
R₁ represents hydrogen, hydroxy, hydroxymethyl, aryl, alkylaryl, amino, alkoxy, carbamoyl or halogen (e.g. fluorine, chlorine, bromine and iodine);
R₂ represents alkyl, C₃-C₇ cycloalkyl ring, C₃-C₇ cycloalkenyl ring, an aryl or a heteroaryl ring having 1-2 hetero atoms selected from the group consisting of oxygen, sulphur and nitrogen atoms; the aryl or heteroaryl rings may be unsubstituted or substituted by one to three substituents independently selected from lower alkyl (C₁-C₄), lower perhaloalkyl (C₁-C₄), cyano, hydroxy, nitro, lower alkoxycarbonyl, halogen, lower alkoxy (C₁-C₄), lower perhaloalkoxy (C₁-C₄), unsubstituted amino, N-lower alkylamino, N-lower alkylamino carbonyl (C₁-C₄);
W represents (CH₂)p, where p represents 0 to 1;
X represents an oxygen, sulphur, NR or no atom, wherein R represents H, alkyl;
Y represents no atom or CHR₅CO, methyl or (CH₂)q; wherein R₅ represents hydrogen, and q represents 0 to 4;
Z represents no atom or NHR₈CO, wherein R represents (CH₂)ᵣ, wherein r represents 0 to 4;
Q represents (CH₂)ₙ wherein n represents 0 to 1;
R₆ and R₇ are independently selected from H, CH₃, COOH, CONH₂, NH₂, CH₂NH₂; and
P is any group which can be used to protect an amino group, for example, benzyl, t-butoxycarbonyl in the presence of a condensing agent to give a protected compound of Formula IV wherein Ar, R₁, R₂, W, X, Y, Z, Q, R₆, R₇ and P are as defined earlier, which on deprotection through reaction with a deprotecting agent in an organic solvent gives an unprotected compound of Formula V wherein Ar, R₁, R₂, W, X Y, Z, Q, R₆ and R₇ are as defined earlier, which is finally N-alkylated or benzylated with a suitable alkylating or benzylating agent L-R₄ wherein L is any leaving group known in the art and R₄ is (i) R₄ represents hydrogen, C₁-C₁₅ saturated or unsaturated aliphatic hydrocarbon (straight chain or branched) groups in which any 1 to 6 hydrogen atoms may be substituted with the group independently selected from halogen, carbonyl, arylalkyl, arylalkenyl, heteroarylalyl or heteroarylalkenyl having 1 to 2 hetero atoms selected from the group consisting of nitrogen, oxygen and sulphur atoms with an option that any 1 to 3 hydrogen atoms on an aryl or heteroaryl ring in said arylalkyl, arylalkenyl, heteroarylalkyl, heteroarylalkenyl rings may be substituted with lower alkyl (C₁-C₄), lower perhaloalkyl (C₁-C₄), cyano, hydroxy, nitro, lower alkoxy carbonyl, halogen, lower alkoxy (C₁-C₄), lower perhalo alkoxy (C₁-C₄), unsubstituted amino, N-lower alkylamino (C₁-C₄), N-lower alkylamino carbonyl (C₁-C₄),
(ii), to give a compound of Formula I.

The reaction of the compound of Formula III with a compound of Formula II to give a compound of Formula IV can be carried out in the presence of a condensing agent, for example, 1-(3-dimethylamino propyl)-3-ethyl carbodiimide hydrochloride (EDC) and 1,8-diazabicyclo[5.4.0]undec-7-ene (DBU).

The reaction of the compound of Formula III with a compound of Formula II to give a compound of Formula IV can be carried out in a suitable solvent, for example, N, N-dimethylformamide, dimethylsulfoxide, toluene and xylene at a temperature ranging from about 0° C. to about 140° C.

The deprotection of the compound of Formula IV to give a compound of Formula V can be carried out with a deprotecting agent, for example, palladium on carbon, trifluoroacetic acid (TFA) and hydrochloric acid.

The deprotection of the compound of Formula IV to give a compound of Formula V can be carried out in a suitable organic solvent, for example, methanol, ethanol, tetrahydrofuran and acetonitrile at a temperature ranging from about 10° C. to about 50° C., for example, from about 25° to about 30° C.

The N-alkylation or benzylation of a compound of Formula V to give a compound of Formula I can be carried out with a suitable alkylating or benzylating agent, L-R₄ wherein L is any leaving group, known in the art) preferably selected from halogen, O-mestyl and O-tosyl group.

The N-alkylation or benzylation of a compound of Formula V to give a compound of Formula I can be carried out in a suitable organic solvent, for example, N,N-dimethylformamide, dimethylsulfoxide, tetrthydrofuran and acetonitrile, at a temperature ranging from about 25° to about 100° C., for example, from about 25° to about 30° C.

In the above scheme, where specific bases, condensing agents, protecting groups, deprotecting agents, N-alkylating/benzylating agents, solvents, catalysts etc. are mentioned, it is to be understood that other bases, condensing agents, protecting groups, deprotecting agents, N-alkylating/benzylating agents, solvents, catalysts etc. known to those skilled in the art may be used. Similarly, the reaction temperature and duration may be adjusted according to the desired needs.

Alternatively, the compounds of the invention may be prepared by condensing compounds of Formula II with an aryl alpha keto ester [Ar(CO)COOR′ wherein R′ denotes a lower alkyl group] and the compounds thus formed may be subsequently reacted with the condensate R″M, wherein R″ groups include groups such as phenyl, C₄₋₆ alkyl etc. and M may be alkali metal or MgX, wherein X is a halogen atom. Alpha keto esters may, in turn, be prepared by following the procedure mentioned in J. Org. Chem., 46, 213 (1981), or Synthetic Communication, 11, 943 (1981).

The compounds of the invention may also be prepared by reacting R″M (wherein M and R″ have the same meaning as described above) with the aryl alpha keto ester [Ar(CO)COOR′ wherein R′ denotes a lower alkyl group] to form an alpha hydroxy ester. This product is further reacted with the compound of Formula II and then the protecting group is removed to give the compound of Formula V.

Suitable salts of compound represented by the Formula I were prepared so as to solubilise the compound in aqueous medium for biological evaluations. Examples of such salts are pharmacologically acceptable salts such as inorganic acid salts (e.g. hydrochloride, hydrobromide, sulphate, nitrate and phosphorate), organic acid salts (e.g. acetate, tartrate, citrate, fumarate, maleate, toluenesulphonate and methanesulphonate). When carboxyl group is included in the Formula I as a substituent, it may be an alkali metal salts (e.g. sodium, potassium, calcium, magnesium, and the like). These salts may be prepared by the usual prior art techniques, such as treating the compound with equivalent amount of inorganic or organic acid or base in a suitable solvent.

Particular compounds which are capable of being produced by Scheme I and shown in Table I include:

TABLE I

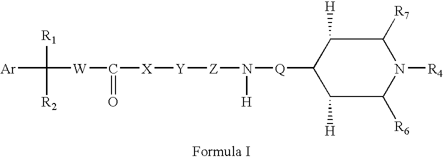

(wherein X is no atom and R₆ = R₇ = H)

| Compound | | | | | | | | |
|---|---|---|---|---|---|---|---|---|
| No. | Ar | R₁ | R₂ | W | Y | Z | Q | R₄ |
| 1 | 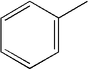 | 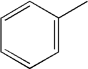 | 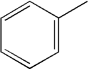 | —CH₂ | — | — | — | 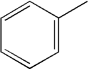 |
| 2 | 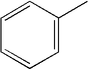 | 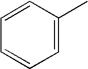 | 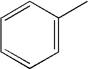 | —CH₂ | — | — | — | 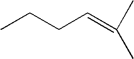 |
| 3 | 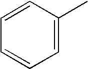 | 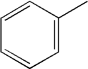 | 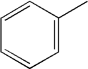 | —CH₂ | — | — | — | 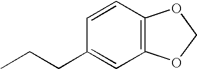 |
| 4 | 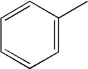 | 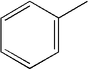 | 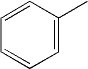 | —CH₂ | — | — | — | 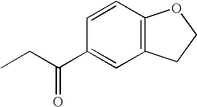 |
| 5 | 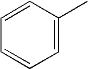 | 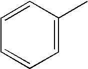 | 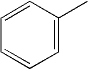 | —CH₂ | CH₂O | NH(CH₂)₂CO | CH₂ | 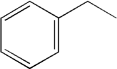 |
| 6 | 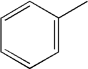 | 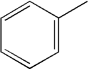 | 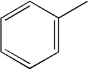 | —CH₂ | CH₂O | NH(CH₂)₂CO | — | 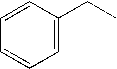 |
| 7 | 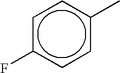 | OH | 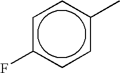 | — | — | — | — | H |
| 8 | 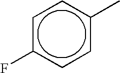 | O(CH₂)₂CH₃ | 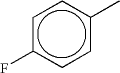 | — | — | — | — | H |

| | |
|---|---|
| COMPOUND NO. | CHEMICAL NAME |
| 1. | (1α, 5α, 6α)-N-[3-benzyl-3-azabicyclo[3.1.0]-hexyl-6-amino-yl]-3,3,3- |
| | triphenylpropionaniide |
| 2. | (1α, 5α, 6α)-N-[3-(4-methyl-3-pentenyl)-3-azabicyclo[3.1.0]-hexyl-6- |
| | amino-yl]-3,3,3-triphenyl propionamide |
| 3. | (1α, 5α, 6α)-N-[3-{2-(3,4-methylenedioxy-phenyl)ethyl}-3-azabicyclo |
| | [3.1.0]-hexyl-6-amino-yl]-3,3,3-triphenylpropionamide |
| 4. | (1α, 5α, 6α)-N-[3-{2-oxo-2-(2,3-dihydrobenzofuran-5-yl)ethyl}-3- |
| | azabicyclo[3.1.0]-hexyl-6-amino-yl]-3,3,3-triphenyl propionamide |
| 5. | (1α, 5α, 6α)-N-[(3-oxo propyl)amino-2-oxoethyl-3-benzyl-3-azabicyclo |
| | [3.1.0]-hexyl-6-aminomethyl]-3,3,3-triphenyl propionamide |
| 6. | (1α, 5α, 6α)-N-[(3-oxo propyl]amino-2-oxoethyl-3-benzyl-3-azabicyclo |
| | [3.1.0]-hexyl-6-amino-yl]-3,3,3-triphenyl propionamide |
| 7. | (1α, 5α, 6α)-N-[3-azabicylo[3.1.0]-hexyl-6-amino-yl]-2-hydroxy-2,2-bis- |
| | 4-fluorophenyl acetamide |
| 8. | (1α, 5α, 6α)-N-[3-azabicyclo[3.1.0]-hexyl-6-amino-yl]-2-propyloxy-2,2- |
| | bis-4-fluorophenyl acetamide |

### EXPERIMENTAL DETAILS

Various solvents such as acetone, methanol, pyridine, ether, tetrahydrofuran, hexane and dichloromethane were dried using various drying reagents according to the procedures well known in the literature. IR spectra were recorded as nujol mulls or a thin neat film on a Perkin Elmer Paragon instrument, Nuclear Magnetic Resonance (NMR) were recorded on a Varian XL-300 MH_{z} instrument using tetramethylsilane as an internal standard.

### Example—1

### Preparation of ((1α,5α,6α)-N-[3-benzyl-3-azabicyclo[3.1.0]-hexyl-6-amino-yl]-3,3,3-triphenylpropionamide (Compound No. 1)

To a solution of triphenylpropionic acid (2 g, 6.6 mmol) and 3-azabicyclo[3.1.0]hexyl-6-amine (prepared following the procedure of T. F. braish et. al., Synlett 1996, 1100 (1.25 g, 6.6 mmol) in dimethylformamide (50 ml), N-methylmorpholine (1.67 g, 16.5 mmol), and 1-hydroxy benzotriazole (894 mg, 6.6 mmol) were added at 0° C. The mixture was warmed to room temperature and stirred for 45 minutes. 1-(3-dimethylaminopropyl)-3-ethylcarbodiimide hydrochloride (1.26 g, 6.6 mmol) was added to it at 0° C. and stirred for 1 h at the same temperature. It was warmed to room temperature and stirred overnight. The reaction was quenched by the addition of water and the organic compound was extracted with ethyl acetate. The aqueous layer was extracted with ethyl acetate and the combined organic layer was washed with water and brine. It was dried (Na₂SO₄) and evaporated to give an off-white solid which was triturated with hexane to give an off-white fine powder. This was filtered off and washed with hexane.

M.P. 178-183° C. ¹H NMR (CDCl₃): 7.31-7.16 (20H, m), 4.60 (1H, m), 3.48 (2H, d), 2.91 (2H, d), 2.75 (1H, s), 2.22 (2H, d), IR (KBr): 1637 cm⁻¹.

### Example—2

### Preparation of (1α,5α,6α)-N-[3-(4-methyl-3-pentenyl)-3-azabicyclo[3.1.0]-hexyl-6-amino-yl]-3,3,3-triphenylpropioamide (Compound No. 2)

To a solution of (1α,5α,6α)-3-azabicyclo[3.1.0]hexyl-6-amino-yl-3,3,3-triphenylpropionamide (which was prepared after debenzylation of compound No. 1 with Pd—C in methanol) (150 mg, 0.39 mmol) in dimethylformamide (5 ml), K₂CO₃ (138 mg, 1 mmol), KI (65 mg, 0.39 mmol) and 4-methyl-3-pentenyl bromide (commercially available) (64 mg, 0.39 mmol) were added and the mixture was stirred at 60-70° C. for 3 h and then at room temperature overnight. The reaction was quenched with water and extracted with ethyl acetate. The organic layer was separated and washed with water, brine, dried (Na₂SO₄) and evaporated to give a crude oil. This was purified with column chromatography over silica gel using dichloromethane-methanol (0-2%) as an eluting solvent.

M.P. 115-28° C. ¹H NMR (CDCl₃): 7.31-7.18 (15H, m), 5.02 (1H, t), 4.62 (1H, m), 3.49 (2H, m), 2.97 (2H, d), 2.62 (1H, s), 2.25 (4H, m), 2.02 (2H, m), 1.65 (3H, s), 1.56 (3H, s), 0.9 (2H, m). IR (KBr): 3255 and 1638 cm⁻¹.

### Example—3

### Preparation of (1α,5α,6α)-N-[3-(2-(3,4-methylenedioxyphenyl)-3-azabicyclo[3.1.0]-hexyl-6-amino-yl]-3,3,3-triphenylpropionamide (Compound No. 3)

To a solution of (1α,5α,6α)-3-azabicyclo[3.1.0]hexyl-6-amino-yl-3,3,3-triphenylpropionamide (which was prepared after debenzylation of compound No. 1 with Pd—C in methanol) (158 mg, 0.41 mmol) in acetonitrile (5 ml), K₂CO₃ (143 mg, ˜1 mmol), KI (69 mg, 0.41 mmol) and 2-(3,4-methylenedioxyphenyl)ethylbromide (which was prepared by reducing commercially available 2-(3,4-methylenedioxy phenyl)-ethnoic acid with lithium aluminum hydride followed by reaction with phosphorous tribromide) (95 mg, 0.41 mmol) were added and the mixture was stirred at 60-70° C. for 2 h and then at room temperature overnight. The reaction was quenched with water and extracted with ethyl acetate. The organic layer was separated and washed with water, brine, dried (Na₂SO₄) and evaporated to give a sticky oil. This was purified with column chromatography over silica gel using dichloromethane-methanol (0-2%) as an eluting solvent, to give the pure product as a white solid.

M.P. 130-133° C. ¹H NMR (CDCl₃): 7.29-7.20 (15H, m), 6.70-6.56 (3H, m), 5.90(2H, s), 4.62(1H, m), 3.71(1H, m), 3.51(2H, m), 3.0(2H, d), 2.52(4H, m), 2.22(2H, d), 0.93(2H, m). IR (KBr): 3292 and 1654 cm⁻¹.

### Example—4

### Preparation of (1α,5α,6α)-N-[3-(2-oxo-2-(2,3-dihydrobenzofuran-5-yl)ethyl)-3-azabicyclo[3.1.0]-hexyl-6-amino-yl]-3,3,3-triphenylpropionamide (Compound No. 4)

To a solution of (1α,5α,6α)-3-azabicyclo[3.1.0]hexyl-6-amino-yl-3,3,3-triphenylpropionamide (which was prepared after debenzylation of compound No. 1 with Pd—C in methanol) (120 mg, 0.31 mmol) in dimethylformamide (5 ml), K₂CO₃ (87 mg, 0.78 mmol), KI (52 mg, 0.31 mmol) and 2-oxo-2-(2,3-dihydrobenzofuran-5-yl)ethylchloride (which was prepared by reacting 3-chloropropionyl chloride with benzofuran) (62 mg, 0.31 mmol) were added and the mixture was stirred at room temperature overnight. The reaction was quenched with water and extracted with ethyl acetate. The organic layer was separated and washed with water, brine, dried (Na₂SO₄) and evaporated to give a crude oil. This was purified with column chromatography over silica gel using dichloromethane-methanol (0-4%) as an eluting solvent to give the pure product as a sticky brown solid.

¹H NMR (CDCl₃): 7.8 (1H, m), 7.26 (16H, m), 6.75 (1H, m), 4.64 (3H, m), 3.65 (2H, m), 3.49 (2H, m), 3.22 (2H, t), 3.05 (2H, d), 2.62 (1H, s), 2.48 (2H, m), 0.91 (2H, m).

### Example—5

### Preparation of (1α,5α,6α)-N-[(3-benzyl-3-azabicyclo[3.1.0]-hexyl-6-aminomethyl)-3-oxopropyl]amino-2-oxoethyl)-3,3,3-triphenylpropionamide (Compound No. 5)

To a suspension of [(3-methoxy-3-oxopropyl)amino-2-oxoethyl]-3,3,3-triphenylpropionamide (434 mg, 0.97 mmol) in CHCl₃ (1 ml) and MeOH (2 ml) was added 10% aq. NaOH solution (2 ml) and the mixture was stirred at RT for 3 h. The mixture was acidified with 1N HCl solution and extracted with CHCl₃. The organic layer was dried and evaporated to give the crude acid (404 mg, 0.91 mmol). To it, 3-benzyl-3-azabicyclo[3.1.0]hexyl-6-aminomethyl (which was synthesized following the procedure of EP 0413455A2) (184 mg, 0.91 mmol) was added and was dissolved in chloroform (4 ml), followed by the addition of 1-(3-dimethylaminopropyl)-3-ethylcarbodiimide hydrochloride (210 mg, 0.91 mmol) and 1-hydroxy benzotriazole (148 mg. 0.91 mmol). The mixture was stirred for 18 h at RT. The mixture was quenched by the addition of saturated aq. NaHCO₃ solution and the organic compound was extracted into chloroform The aqueous layer was extracted with chloroform and the combined organic layer was washed with water, brine, dried (Na₂SO₄) and evaporated to give the crude product as a yellow oil. This was purified with column chromatography over silica gel using dichloromethane-methanol (0-5%) as an eluting solvent to give the pure product as a white solid.

M.P. 50-70° C. ¹H NMR (CDCl₃): 7.35-7.18 (20H, m), 6.26 (1H, m), 5.75 (1H, m), 5.59 (1H, m), 3.63-3.51 (6H, m), 3.38 (2H, m), 3.02 (4H, m), 2.39-2.27 (4H, m), 1.41 (1H, m), 1.27 (1H, m), 0.88 (1H, m). IR (KBr): 3303, 1654 cm⁻¹.

### Example—6

### Preparation of (1α,5α,6α)-N-[((3-benzyl-3-azabicyclo[3.1.0]-hexyl-6-amino-yl)-3-oxopropyl]amino-2-oxoethyl)-3,3,3-triphenylpropionamide (Compound No. 6)

To a suspension of [(3-methoxy-3-oxopropyl)amino-2-oxoethyl]-3,3,3-triphenyl propionamide (140 mg, 0.31 mmol) in CHCl₃ (1 ml) and MeOH (2 ml) was added 10% aq. NaOH solution (2 ml) and the mixture was stirred at RT for 4 h. The mixture was acidified with IN HCl solution and extracted with CHCl₃. The organic layer was dried and evaporated to give the crude acid. To it, 3-benzyl-3-azabicyclo[3.1.0]hexyl-6-amine (which was prepared following the procedure of T. F. Braish et. al., Synlett 1996, 1100) (59 mg, 0.31 mmol) was added and was dissolved in chloroform (4 ml), followed by the addition of 1-(3-dimethylaminopropyl)-3-ethylcarbodiimide hydrochloride (73 mg, 0.31 mmol) and 1-hydroxy benzotriazole (51 mg, 0.31 mmol). The mixture was stirred for 18 h at RT. The mixture was quenched by the addition of saturated aq. NaHCO₃ solution and the organic compound was extracted into chloroform. The aqueous layer was extracted with chloroform and the combined organic layer was washed with water, brine, dried (Na₂SO₄) and evaporated to give the crude product. This was purified with column chromatography over silicagel using dichloromethane-methanol (0-5%) as an eluting solvent to give the product as a white solid.

M.P.>150° C. (dec) ¹H NMR (CDCl₃): 7.31-7.23 (20H, m), 6.27 (1H, m), 5.82 (1H, m), 5.56 (1H, m), 3.65-3.37 (8H, m), 3.09 (3H, m), 2.40 (2H, m), 2.24 (2H, m), 1.47 (2H, m).

### Example—7

### Preparation of (1α,5α,6α)-N-[3-azabicyclo[3.1.0]-hexyl-6-amino-yl]-2-hydroxy-2,2-bis-4-fluorophenyl acetamide (Compound No. 7)

Step a: Preparation of 2-hydroxy-2,2-bis-(4-fluorophenyl)acetic acid

(i) Preparation of 1,2 Bis(4-fluorophenyl)-2-hydroxy ethanone.

To a solution of 4-fluorobenzaldehyde(24.8 g, 200 mmole) in ethanol (30 ml), NaCN (2.13 g, 43.5 mmol) in water (20 ml) was added and the resulting solution was refluxed for 1 hour. It was cooled to 0° C. and diluted with water. The solid so separated was filtered and washed with cold water thoroughly and used as such in the next step.

(ii) Preparation of 1,2-Bis (4-fluorophenyl)-2-oxo-ethanone

To the compound obtained in the above step was added conc. nitric acid (40 ml) and the resulting solution was refluxed for 4 hours. It was cooled and poured on to chilled water (500 ml) under stirring and the solid so separated was filtered, washed with water and dried to give the title compound in 63% yield.

(iii) Preparation of 2-hydroxy-2,2-bis-(4-fluorophenyl)acetic acid.

To a solution of KOH (21.0 gm) in water (42.0 ml), ethanol (54.0 ml) and the compound obtained from the above step (25.0 g, 101 mmol) was added and the resulting solution was refluxed for 30 minutes and poured into a glass plate and left overnight at RT. The semisolid obtained was dissolved in water (400 ml) and washed with ethyl acetate. The pH of the aqueous layer was adjusted to acidic with 50% HCl, and extracted with ethyl acetate. The ethyl acetate layer was dried over anhydrous Na₂SO₄ and concentrated to give the title compound in 45% yield (12.0 g, 45 mmol).

Step b: Preparation of (1α,5α,6α)-N-[3-benzyl-3-azabicylo[3.1.0]-hexyl-6-amino-yl]-2-hydroxy-2,2-bis-4-fluorophenyl acetamide

To a solution of (1α,5α,6α)-N-[3-benzyl-3-azabicylo[3.1.0]hexyl amine (prepared following the procedure of T. F. Braish et. al., Synlett 1996, 1100) in toluene, 1,8-diazabicylo[5.4.0]undec-7-ene (DBU) and 2-hydroxy-2,2-bis-(4-fluoro phenyl)acetic acid were added. The reaction mixture was refluxed for 14 hours and purified by column chromatography using ethyl acetate in hexane as an eluent to give the title compound in 58% yield.

Step c: Preparation of (1α,5α,6α)-N-[3-azabicylo[3.1.0]-hexyl-6amino-yl]-2-hydroxy-2,2-bis-4-fluorophenyl acetamide.

To a solution of compound obtained in step b in methanol, 10% Pd—C was added and the resulting solution was hydrogenated at 50 psi and at RT for 2 hours. The reaction mixture was filtered through a bed of hyflo and was washed with methanol. The filtrate was concentrated to give the title compound as an oil in 90% yield.

IR(KBr): 1651.4 cm^{-1 1}HNMR(CDCl₃):δ 7.36-7.47 (m, 4H), 7.01-7.10 (m, 4H), 3.36-3.51 (m, 4H), 2.70 (s, 1H), 1.94-2.22 (s, 2H)

### Example—8

### Preparation of (1α,5α,6α)-N-[3-azabicylo[3.1.0]-hexyl-6-amino-yl]-2-propyloxy-2,2-bis-4-fluorophenyl acetamide (Compound No. 8)

Step a: Preparation of 2-propyloxy-2,2-bis-(4-fluorophenyl)acetic acid

(i) Preparation of 2-hydroxy-2,2-bis-(4-fluorophenyl) acetic acid

This was synthesized as described in step a of Example—7.

(ii) Preparation of 2-hydroxy acetic acid 2,2-bis-(4-fluorophenyl) ethyl ester

To a solution of the compound obtained in the above step (50 g, 18.9 mmol) in ethanol (100.0 ml) at 0° C., thionyl chloride (5.0 ml) was added and the resulting solution was refluxed for 4 hr. Ethanol was concentrated under vacuum and the residue was purified by column chromatography using 20% ethyl acetate in hexane to give the title compound as liquid in 91% (5.08 g, 17.2 mmol) yield.

(iii) Preparation of 2,2-bis-(4-fluorophenyl)-2-propoxy acetic acid ethyl ester

To a solution of NaH (0.72 g, 15.42 mmol) in DMF (1.0 ml) at 0° C., the hydroxy ester (1.5 g, 5.14 mmol) in DMF (5.0 ml) was added and stirred at RT for 30 minutes. The reaction mixture was cooled to 0° C. and bromo propane (0.95 g, 7.7 mmol) was added and stirred for 4 hr. at RT, diluted with water, extracted with ethyl acetate, dried and concentrated. The residue was purified by column chromatography using 10% ethyl acetate in hexane to get the title compound as a liquid in 46% (0.79 g, 2.36 mmol) yield.

(iv) Preparation of 2-propyloxy-2,2-bis-(4-fluorophenyl) acetic acid

To a solution of the ester obtained in the above step (0.7 g, 2 mmol) in methanol (20.9 ml), 1N LiOH (2.0 ml) was added and the reaction mixture was stirred at RT for 12 hr. Methanol was concentrated under vacuum, the residue was taken in water (50.0 ml) and washed with ethyl acetate. The aqueous layer was neutralized with acetic acid and extracted with ethyl acetate, dried and concentrated under vacuum to give the title compound as an oil in 47% (0.3 g, 0.94 mmol) yield.

¹HNMR (CDCl₃):δ 7.44-7.49 (m, 4H), 7.04-7.09 (m, 4H), 4.21-4.23 (m, 2H), 3.20-3.34 (m, 4H), 3.05-3.11 (m, 2H), 2.33-2.72 (m, 3H), 1.32-1.69 (m, 17H), 0.97 (t, J=6 Hz, 3H),

Step b: Preparation of (1α,5α,6α)-N-(3-benzyl-3-azabicyclo[3.1.0]hexyl-6-amino-yl]-2-propyloxy-2,2-bis-4-fluorophenyl acetamide

To a solution of (1α,5α,6α)-N-[3-benzyl-3-azabicyclo[3.1.0]hexyl amine prepared following the procedure of T. F. Braish et. al., Synlett 1996, 1100) in toluene, 1,8-diazabicylo[5.4.0]undec-7-ene (DBU) and 2-propyloxy-2,2-bis-(4-fluorophenyl) acetic acid was added. The reaction mixture was refluxed for 14 hours, cooled and absorbed directly onto silica gel and purified by column chromatography by using ethyl acetate in hexane as an eluent mixture to give the title compound in 60% yield.

Step c: Preparation of (1α,5α,6α)-N-[3-azabicylo[3.1.0]-hexyl-6-amino-yl]-2-propyloxy-2,2-bis-4-fluorophenyl acetamide

To a solution of compound obtained in step b in methanol, 10% Pd—C was added and the resulting solution was hydrogenated at 50 psi and at RT for 2 hours. The reaction mixture was filtered through a bed of hyflo and was washed with methanol. The filtrate was concentrated to give the title compound as an oil in 90% yield.

¹HNMR (CDCl₃):δ 7.32-7.37 (m, 4H), 6.99-7.04 (m, 4H), 3.35 (d, J=12 Hz, 2H), 3.19-3.23 (4m, 2H), 2.91-2.96 (m, 2H), 2.69 (s, 1H), 1.75 (s, 2H), 1.51-1.63 (m, 2H), 0.86-0.91 (m, 3H);

Biological Activity

Radioligand Binding Assays:

The affinity of test compounds for M₂ and M₃ muscarinic receptor subtypes was determined by [³H]-N-methylscopolamine binding studies using rat heart and submandibular gland respectively as described by Moriya et al., (Life Sci., 1999; 64 (25): 2351-2358).

Membrane preparation: Submandibular glands and heart were isolated and placed in ice cold homogenising buffer (EPES 20 mM, 10 mM EDTA, pH 7.4) immediately after sacrifice. The tissues were homogenised in 10 volumes of homogenising buffer and the homogenate was filtered through two layers of wet gauze and filtrate was centrifliged at 500 g for 10 min. The supernatant was subsequently centrifuged at 40,000 g for 20 min. The pellet thus obtained was resuspended in same volume of assay buffer (HEPES 20 mM, EDTA 5 mM, pH 7.4) and were stored at −70° C. until the time of assay.

Ligand binding assay: The compounds were dissolved and diluted in DMSO. The membrane homogenates (150-250 μg protein) were incubated in 250 μl of assay buffer (HEPES 20 mM, pH 7.4) at 24-25° C. for 3 h. Non-specific binding was determined in the presence of 1 μM atropine. The incubation was terminated by vaccum filtration over GF/B fiber filters(Wallac). The filters were then washed with ice cold 50 mM Tris HCl buffer (pH 7.4). The filter mats were dried and bound radioactivity retained on filters was counted. The IC₅₀ & Kd were estimated by using the non-linear curve fitting program using G Pad Prism software. The value of inhibition constant Ki was calculated from competitive binding studies by using Cheng & Prusoff equation (Biochem Pharmacol, 1973,22: 3099-3108), Ki=IC₅₀/(1+L/Kd), where L is the concentration of [³H]NMS used in the particular experiment.
pKi=−log Ki
Functional Experiments Using Isolated Rat Bladder:
Methodology:

Animals were euthanized by overdose of urethane and whole bladder was isolated and removed rapidly and placed in ice cold Tyrode buffer with the following composition (mMol/L) NaCl 137; KCl 2.7; CaCl₂ 1.8; MgC₂ 0.1; NaHCO₃ 11.9, NaH₂PO₄ 0.4; Glucose 5.55 and continuously gassed with 95% O₂ and 5% CO₂

The bladder was cut into longitudinal strips (3 mm wide and 5-6 mm long) and mounted in 10 ml organ baths at 30° C., with one end connected to the base of the tissue holder and the other end connected to a polygraph through a force displacement transducer. Each tissue was maintained at a constant basal tension of 2 g and allowed to equilibrate for 1 hour during which the PSS was changed every 15 min. At the end of equilibration period, the stabilization of the tissue contractile response was assessed with 1 μmol/L of Carbachol consecutively for 2-3 times. Subsequently, a cumulative concentration response curve to carbachol (10⁻⁹ mol/L to 3×10⁻⁵ mol/L) was obtained. After several washes, once the baseline was achieved, cumulative concentration response curve was obtained in presence of NCE (NCE added 20 min prior to the second CRC).

The contractile results were expressed as % of control E max. ED50 values were calculated by fitting a non-linear regression curve (Graph Pad Prism) PKB values were calculated by the formula pKB=−log [(molar concentration of antagonist/(dose ratio-1))] where,

dose ratio=ED50 in the presence of antagonist/ED50 in the absence of antagonist.

The in-vitro testing data is depicted below in Table II:

TABLE II
Receptor Binding Assay

| | M₂ | M₃ |
|---|---|---|
| Compound No. | (pKi) | (pKi) |
| 1 | <6 | <6 |
| 2 | <6 | <6 |
| 3 | <6 | <6 |
| 4 | <6 | <6 |
| 5 | <6 | <6 |
| 6 | <6 | <6 |
| 7 | <6 | <6 |
| 8 | <6 | <6 |

While the present invention has been described in terms of its specific embodiments, certain modifications and equivalents will be apparent to those skilled in the art and are intended to be included within the scope of the present invention.

We claim

1. Compounds having the structure of Formula I 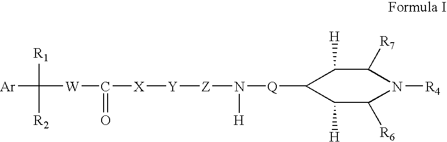
and their pharmaceutically acceptable salts, pharmaceutically acceptable solvates, esters, enantiomers, diastereomers, N-oxides, polymorphs, prodrugs, or metabolites, wherein
Ar represents an aryl or a heteroaryl ring having 1-2 hetero atoms selected from the group consisting of oxygen, sulphur and nitrogen atoms, the aryl or heteroaryl rings may be unsubstituted or substituted by one to three substituents independently selected from lower alkyl (C₁-C₄), lower perhaloalkyl (C₁-C₄), cyano, hydroxy, nitro, halogen (e.g. F, Cl, Br, I), lower alkoxy (C₁-C₄), lower perhaloalkoxy (C₁-C₄), unsubstituted amino, N-lower alkyl (C₁-C₄) amino or lower alkyl (C₁-C₄) amino carbonyl;
R₁ represents hydrogen, hydroxy, hydroxymethyl, aryl, alkylaryl, amino, alkoxy, carbamoyl or halogen (e.g. fluorine, chlorine, bromine and iodine);
R₂ represents alkyl, C₃-C₇ cycloalkyl ring, C₃-C₇ cycloalkenyl ring, an aryl or a heteroaryl ring having 1-2 hetero atoms selected from the group consisting of oxygen, sulphur and nitrogen atoms; the aryl or heteroaryl rings may be unsubstituted or substituted by one to three substituents independently selected from lower alkyl (C₁-C₄), lower perhaloalkyl (C₁-C₄), cyano, hydroxy, nitro, lower alkoxycarbonyl, halogen, lower alkoxy (C₁-C₄), lower perhaloalkoxy (C₁-C₄), unsubstituted amino, N-lower alkylamino, N-lower alkylamino carbonyl (C₁-C₄);
W represents (CH₂)p, where p represents 0 to 1;
X represents an oxygen, sulphur, NR or no atom, wherein R represents H, alkyl;
Y represents no atom or CHR₅CO, methyl or (CH₂)q; wherein R₅ represents hydrogen and q represents 0 to 4;
Z represents no atom or NHR₈CO, wherein R₈ represents (CH₂)ᵣ, wherein r represents 0 to 4;
Q represents (CH₂)ₙ wherein n represents 0 to 1;
R₆ and R₇ are independently selected from H, CH₃, COOH, CONH₂, NH₂, CH₂NH₂; and
R₄ represents hydrogen, C₁-C₁₅ saturated or unsaturated aliphatic hydrocarbon (straight chain or branched) groups in which any 1 to 6 hydrogen atoms may be substituted with the group independently selected from halogen, carbonyl, arylalkyl, arylalkenyl, heteroarylalkyl or heteroarylalkenyl having 1 to 2 hetero atoms selected from the group consisting of nitrogen, oxygen and sulphur atoms with an option that any 1 to 3 hydrogen atoms on an aryl or heteroaryl ring in said arylalkyl, arylalkenyl, heteroarylalkyl, heteroarylalkenyl rings may be substituted with lower alkyl (C₁-C₄), lower perhaloalkyl (C₁-C₄), cyano, hydroxy, nitro, lower alkoxy carbonyl, halogen, lower alkoxy (C₁-C₄), lower perhalo alkoxy (C₁-C₄), unsubstituted amino, N-lower alkylamino (C₁-C₄), N-lower alkylamino carbonyl (C₁-C₄).

2. A compound selected from the group consisting of:
(1α,5α,6α)-N-[3-benzyl-3-azabicyclo[3.1.0]-hexyl-6-amino-yl]-3,3,3-triphenylpropionamide (Compound No. 1)
(1α,5α,6α)-N-[3-(4-methyl-3-pentenyl)-3-azabicyclo[3.1.0]-hexyl-6-amino-yl]-3,3,3-triphenyl propionamide (Compound No. 2)
(1α,5α,6α)-N-[3-{2-(3,4-methylenedioxy-phenyl)ethyl}-3-azabicyclo [3.1.0]-hexyl-6-amino-yl]-3,3,3-triphenylpropionamide (Compound No. 3)
(1α,5α,6α)-N-[3-{2-oxo-2-(2,3-dihydrobenzofuran-5-yl)ethyl}-3-azabicyclo[3.1.0]-hexyl-6-amino-yl]-3,3,3-triphenyl propionamide (Compound No. 4)
(1α,5α,6α)-N-[(3-oxo propyl)amino-2-oxoethyl3-benzyl-3-azabicyclo[3.1.0]-hexyl-6-aminomethyl]-3,3,3-triphenyl propionamide (Compound No. 5)
(1α,5α,6α)-N-[(3-oxo propyl)amino-2-oxoethyl3-benzyl-3-azabicyclo[3.1.0]-hexyl-6-amino-yl]-3,3,3-triphenyl propionamide (Compound No. 6)
(1α,5α,6α)-N-[3-azabicylo[3.1.0]-hexyl-6-amino-yl]-2-hydroxy-2,2-bis-4-fluorophenyl acetamide (Compound No. 7)
(1α,5α,6α)-N-[3-azabicyclo[3.1.0]-hexyl-6-amino-yl]-2-propyloxy-2,2-bis-4-fluorophenyl acetamide (Compound No. 8)

3. A pharmaceutical composition comprising a therapeutically effective amount of a compound as defined in claim 1 or 2 optionally together with pharmaceutically acceptable carriers, excipients or diluents.

4. A method for treatment or prophylaxis of an animal or a human suffering from a disease or disorder of the respiratory, urinary and gastrointestinal systems, wherein the disease or disorder is mediated through muscarinic receptor, comprising admistering to said animal or human, a therapeutically effective amount of a compound having the structure of Formula I, 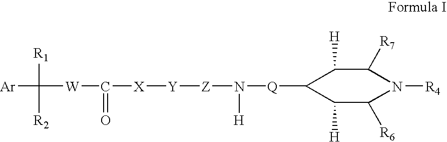
or its pharmaceutically acceptable salts, pharmaceutically acceptable solvates, esters, enantiomers, diastereomers, N-oxides, polymorphs, prodrugs or metabolites, wherein
Ar represent an aryl or a heteroaryl ring having 1-2 hetero atoms selected from the group consisting of oxygen, sulphur and nitrogen atoms, the aryl or heteroaryl rings may be unsubstituted or substituted by one to three substituents independently selected from lower alkyl (C₁-C₄), lower perhaloalkyl (C₁-C₄), cyano, hydroxy, nitro, halogen (e.g. F, Cl, Br, I), lower alkoxy (C₁-C₄), lower perhaloalkoxy (C₁-C₄), unsubstituted amino, N-lower alkyl (C₁-C₄) amino or lower alkyl (C₁-C₄) amino carbonyl;
R₁ represents hydrogen, hydroxy, hydroxymethyl, aryl, alkylaryl, amino, alkoxy, carbamoyl or halogen (e.g. fluorine, chlorine, bromine and iodine);
R₂ represents alkyl, C₃-C₇ cycloalkyl ring, C₃-C₇ cycloalkenyl ring, an aryl or a heteroaryl ring having 1-2 hetero atoms selected from the group consisting of oxygen, sulphur and nitrogen atoms; the aryl or heteroaryl rings may be unsubstituted or substituted by one to three substituents independently selected from lower alkyl (C₁-C₄), lower perhaloalkyl (C₁-C₄), cyano, hydroxy, nitro, lower alkoxycarbonyl, halogen, lower alkoxy (C₁-C₄), lower perhaloalkoxy (C₁-C₄), unsubstituted amino, N-lower alkylamino, N-lower alkylamino carbonyl (C₁-C₄);
W represents (CH₂)p, where p represents 0 to 1;
X represents an oxygen, sulphur, NR or no atom, wherein R represents H, alkyl;
Y represents no atom or CHR₅CO, methyl or (CH₂)q; wherein R₅ represents hydrogen and q represents 0 to 4;
Z represents no atom or NHR₈CO, wherein R₈ represents (CH₂)ᵣ, wherein r represents 0 to 4;
Q represents (CH₂)ₙ wherein n represents 0 to 1;
R₆ and R₇ are independently selected from H, CH₃, COOH, CONH₂, NH₂, CH₂NH₂; and
R₄ represents hydrogen, C₁-C₁₅ saturated or unsaturated aliphatic hydrocarbon (straight chain or branched) groups in which any 1 to 6 hydrogen atoms may be substituted with the group independently selected from halogen, carbonyl, arylalkyl, arylalkenyl, heteroarylalkyl or heteroarylalkenyl having 1 to 2 hetero atoms selected from the group consisting of nitrogen, oxygen and sulphur atoms with an option that any 1 to 3 hydrogen atoms on an aryl or heteroaryl ring in said arylalkyl, arylalkenyl, heteroarylalkyl, heteroarylalkenyl rings may be substituted with lower alkyl (C₁-C₄), lower perhaloalkyl (C₁-C₄), cyano, hydroxy, nitro, lower alkoxy carbonyl, halogen, lower alkoxy (C₁-C₄), lower perhalo alkoxy (C₁-C₄), unsubstituted amino, N-lower alkylamino (C₁-C₄), N-lower alkylamino carbonyl (C₁-C₄).

5. The method according to claim 4 wherein the disease or disorder is urinary incontinence, lower urinary tract symptoms (LUTS), bronchial asthma, chronic obstructive pulmonary disorders (COPD), pulmonary fibrosis, irritable bowel syndrome, obesity, diabetes or gastro intestinahyperkinesis.

6. The method for treatment or prophylaxis of an animal or a human suffering from a disease or disorder of the respiratory, urinary and gastrointestinal systems, wherein the disease or disorder is mediated through muscarinic receptors, comprising administering to the animial or human, a therapeutically effective amount of the pharmaceutical composition according to claim 3.

7. The method according to claim 6 wherein the disease or disorder is urinary incontinence, lower urinary tract symptoms (LUTS), bronchial asthma, chronic obstructive pulmonary disorders (COPD), pulmonary fibrosis, irritable bowel syndrome, obesity, diabetes and gastro intestinahyperkinesis.

8. A process of preparing a compound having the structure of Formula I, 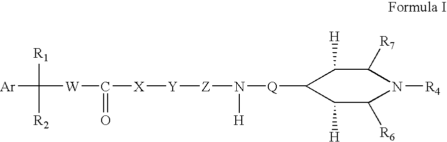
and its pharmaceutically acceptable salts, pharmaceutically acceptable solvates, esters, enantiomers, diastereomers, N-oxides, polymorphs, prodrugs, or metabolites, wherein
Ar represents an aryl or a heteroaryl ring having 1-2 hetero atoms selected from the group consisting of oxygen, sulphur and nitrogen atoms, the aryl or heteroaryl rings may be unsubstituted or substituted by one to three substituents independently selected from lower alkyl (C₁-C₄), lower perhaloalkyl (C₁-C₄), cyano, hydroxy, nitro, halogen (e.g. F, Cl, Br, I), lower alkoxy (C₁-C₄), lower perhaloalkoxy (C₁-C₄), unsubstituted amino, N-lower alkyl (C₁-C₄) amino or lower alkyl (C₁-C₄) amino carbonyl;
R₁ represents hydrogen, hydroxy, hydroxymethyl, aryl, alkylaryl, amino, alkoxy, carbamoyl or halogen (e.g. fluorine, chlorine, bromine and iodine);
R₂ represents alkyl, C₃-C₇ cycloalkyl ring, C₃-C₇ cycloalkenyl ring, an aryl or a heteroaryl ring having 1-2 hetero atoms selected from the group consisting of oxygen, sulphur and nitrogen atoms; the aryl or heteroaryl rings may be unsubstituted or substituted by one to three substituents independently selected from lower alkyl (C₁-C₄), lower perhaloalkyl (C₁-C₄), cyano, hydroxy, nitro, lower alkoxycarbonyl, halogen, lower alkoxy (C₁-C₄), lower perhaloalkoxy (C₁-C₄), unsubstituted amino, N-lower alkylamino, N-lower alkylamino carbonyl (C₁-C₄);
W represents (CH₂)p, where p represents 0 to 1;
X represents an oxygen, sulphur, NR or no atom, wherein R represents H, alkyl;
Y represents no atom or CHR₅CO, methyl or (CH₂)q; wherein R₅ represents hydrogen and q represents 0 to 4;
Z represents no atom or NHR₈CO, wherein R₈ represents (CH₂)ᵣ, wherein r represents 0 to 4;
Q represents (CH₂)ₙ wherein n represents 0 to 1;
R₆ and R₇ are independently selected from H, CH₃, COOH, CONH₂, NH₂, CH₂NH₂; and
R₄ represents hydrogen, C₁-C₁₅ saturated or unsaturated aliphatic hydrocarbon (straight chain or branched) groups in which any 1 to 6 hydrogen atoms may be substituted with the group independently selected from halogen, carbonyl, arylalkyl, arylalkenyl, heteroarylalkyl or heteroarylalkenyl having 1 to 2 hetero atoms selected from the group consisting of nitrogen, oxygen and sulphur atoms with an option that any 1 to 3 hydrogen atoms on an aryl or heteroaryl ring in said arylalkyl, arylalkenyl, heteroarylalkyl, heteroarylalkenyl rings may be substituted with lower alkyl (C₁-C₄), lower perhaloalkyl (C₁-C₄), cyano, hydroxy, nitro, lower alkoxy carbonyl halogen, lower alkoxy (C₁-C₄), lower perhalo alkoxy (C₁-C₄), unsubstituted amino, N-lower alkylamino (C₁-C₄), N-lower alkylamino carbonyl (C₁-C₄),
comprising
(a) condensing a compound of Formula III with a compound of Formula II 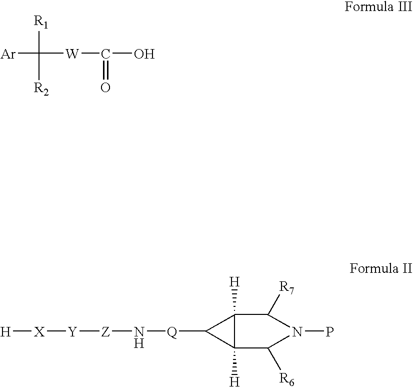
wherein Ar, R₁, R₂, W, X, Y, Z, Q, R₆, and R₇ are the same as defined earlier, to give a protected compound of Formula IV wherein Ar, R₁, R₂, W, X, Y, Z, Q are as defined earlier and P is a protecting group for an amino group, 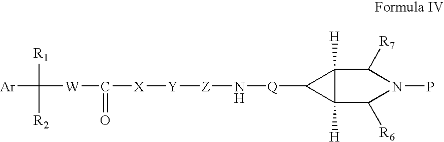
(b) deprotecting the compound of Formula IV in the presence of a deprotecting agent to give an unprotected compound of Formula V wherein Ar, R₁, R₂, W, X, Y, Z, and Q are as defined earlier, and 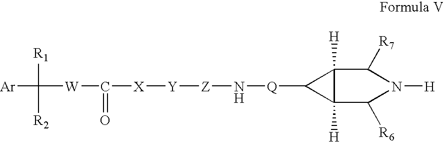
(c) the compound of Formula V with a suitable N-alkylating or benzylating agent to give a compound of Formula I wherein Ar, R₁, R₂, W, X, Y, Z, Q, R₄, R₆ and R₇ are as defined earlier.

9. The process according to claim 8 wherein P is selected from the group consisting of benzyl and t-butyloxy carbonyl group.

10. The process according to claim 8 wherein the reaction of a compound of Formula II with a compound of Formula III to give a compound of Formula IV is carried out in the presence of a condensing agent selected from the group consisting of 1-(3-dimethyl amino propyl)-3-ethyl carbodiimide hydrochloride (EDC) and 1,8-diazabicyclo[5.4.0]undec-7-ene (DBU).

11. The process according to claim 8 wherein the reaction of a compound of Formula IH with a compound of Formula III to give a compound of Formula IV is carried out in a solvent selected from the group consisting of N,N-dimethyl formamide, dimethylsulfoxide, toluene and xylene.

12. The process according to claim 8 wherein the reaction of a compound of Formula II with a compound of Formula III is carried out at a temperature ranging from about 0° C. to about 140° C.

13. The process according to claim 8 wherein the deprotection of a compound of Formula IV to give a compound of Formula V is carried out with a deprotecting agent selected from the group consisting of palladium on carbon, trifluoroacetic acid (TFA) and hydrochloric acid.

14. The process according to claim 8 wherein the deprotection of a compound of Formula IV to give a compound of Formula V is carried out in a solvent selected from the group consisting of methanol, ethanol, tetrahydrofuran and acetonitrile.

15. The process according to claim 8 wherein the N-alkylation or benzylation of a compound of Formula V to give a compound of Formula I is carried out with an alkylating or benzylating agent, L-R₄ wherein L is any leaving group and R₄ is as defined earlier.

16. The process according to claim 15 wherein the leaving group is selected from the group consisting of halogen, O-mestyl and O-tosyl groups.

17. The process according to claim 15 wherein the N-alkylation or benzylation of a compound of Formula V to give a compound of Formula I is carried out in a solvent selected from the group consisting of N,N-dimethylformamide, dimethylsulfoxide, tetrahydrofuran and acetonitrile.
